(19) United States
(12) Patent Application Publication
(10) Pub. No.: US 2013/0317037 A1
(43) Pub. Date: Nov. 28, 2013

(54) METHOD OF ADMINISTRATION AND TREATMENT

(71) Applicants: Kurtis Earl Bachman, Collegeville, PA (US); Joel David Greshock, Collegeville, PA (US)

(72) Inventors: Kurtis Earl Bachman, Collegeville, PA (US); Joel David Greshock, Collegeville, PA (US)

(73) Assignee: GlaxoSmithKline Intellectual Property Development Limited, Middlesex (GB)

(21) Appl. No.: 13/885,804

(22) PCT Filed: Nov. 16, 2011
(86) PCT No.: PCT/US11/60958

§ 371 Date: May 16, 2013

Related U.S. Application Data
(60) Provisional application No. 61/414,115, filed on Nov. 16, 2010.

(51) Int. Cl. A61K 31/501; A61K 45/06

(52) U.S. Cl. CPC A61K 31/501; A61K 45/06
USPC 514/252.04

(57) ABSTRACT

The present invention provides a method of treating a human with cancer comprising detecting at least one mutation in a PIK3CA gene or at least one mutant protein encoded by said PIK3CA gene from at least one first sample from said human and administering to said human an effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition if said at least one sample has at least one mutant PI3K protein or a mutation in the PIK3CA gene.

### FIELD OF THE INVENTION

This invention relates to the administration of drug and methods of treating cancer patients.

### BACKGROUND OF THE INVENTION

The expanding development and use of targeted therapies for cancer treatment reflects an increasing understanding of key oncogenic pathways, and how the targeted perturbation of these pathways corresponds to clinical response. Difficulties in predicting efficacy to targeted therapies is likely a consequence of the limited global knowledge of causal mechanisms for pathway deregulation (e.g. activating mutations, amplifications). Pre-clinical translational research studies for oncology therapies focuses on determining what tumor type and genotypes are most likely to benefit from treatment. Treating selected patient populations may help maximize the potential of a therapy. Pre-clinical cellular response profiling of tumor models has become a cornerstone in development of novel cancer therapeutics. Efforts to predict clinical efficacy using cohorts of in vitro tumor models have been successful (e.g. EGFR inhibitors are selectively useful in those tumors harboring EGFR mutations). Thus, expansive panels of diverse tumor derived cell lines could recapitulate an ‘all comers’ efficacy trial; thereby identifying which histologies and specific tumor genotypes are most likely to benefit from treatment. Numerous specific molecular markers are now used to identify patients most likely to benefit in a clinical setting. For example, in vitro, imatinib selectively kills cells with the activated BCR-ABL gene fusion (Carroll et al., 1997), while lapatinib preferentially inhibits proliferation of Her2 over expressing cells (Rusnak et al., 2007). Both have achieved commercial success, benefiting patients with tumors harboring these genetic aberrations.

The phosphoinositide 3-kinase (PI3K) pathway is among the most commonly activated pathways in human cancer. The function and importance of this pathway in tumorigenesis and tumor progression is well established (Samuels & Ericson. Curr. Opp in Oncology, 2006. 18: 77-82). PI3K-AKT signaling appears to be a pivotal modulator of cell survival, proliferation and metabolism. This includes the activation of mammalian target of rapamycin (mTOR), a PI3K protein family member and direct regulator of cell growth and translation. Thus, the deregulation of PI3K/AKT/mTOR signaling in tumors contributes to a cellular phenotype that demonstrates numerous hallmarks of malignancies, which includes unlimited reproductive potential and the evasion of apoptosis (Hanahan & Weinberg, Cell. 2000. 100:57-70).

Activation of this pathway often occurs indirectly by the activation of receptor tyrosine kinases or the inaction of the PTEN tumor suppressor. Also, direct activation of PI3K can be the result of activating mutations in PIK3CA, the gene that encodes the p110α catalytic subunit of PI3Kα. Three ‘hot spot’ mutations have been identified in PIK3CA, two located in the helical domain, E542K and E545K, and one in the kinase domain, H1047R. These and other mutations found in PIK3CA have been shown to activate the lipid kinase activity of PI3Kα, induce activation of signaling pathways, and promote transformation cells in culture. Her2 (also known as ERBB2) is a cell membrane surface-bound receptor tyrosine kinase and a member of the epidermal growth factor receptor family. Functionally Her2 is a component of signal transduction pathways that modulate cell growth and differentiation. Her2, a proto-oncogene, is activated in ˜15-20% of breast cancers is also known to be an upstream activator of PI3K/AKT signal transduction, among other oncogenic pathways.

2,4-difluoro-N-{2-(methyloxy)-5-[4-(4-pyridazinyl)-6-quinolinyl]-3-pyridinyl}benzenesulfonamide (herein after Compound B), or a pharmaceutically acceptable salt thereof, is disclosed and claimed, along with pharmaceutically acceptable salts thereof, as being useful as an inhibitor of PI3K activity, particularly in treatment of cancer, in International Application No. PCT/US2008/063819, having an International filing date of May 16, 2008; International Publication Number WO 2008/144463 and an International Publication date of Nov. 27, 2008, the entire disclosure of which is hereby incorporated by reference, Compound B is the compound of example 345. Compound B can be prepared as described in International Application No. PCT/US2008/063819.

Compound B is being tested in human as a new cancer treatment. It is desirable to identify genotypes that are more likely to respond to Compound B.

### SUMMARY OF THE INVENTION

The present invention provides a method of treating a human with cancer comprising detecting at least one mutation in a PIK3CA gene or at least one mutant protein encoded by said PIK3CA gene from at least one first sample from said human and administering to said human an effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition if said at least one sample has at least one mutant PI3K protein or a mutation in the PIK3CA gene.

### DETAILED DESCRIPTION OF THE INVENTION

The present invention also relates to a method of treating a human with cancer comprising detecting at least one mutation in a PIK3CA gene or at least one mutant protein encoded by said PIK3CA gene from at least one first sample from said human and administering to said human an effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition if said at least one sample has at least one mutant PI3K protein or a mutation in the PIK3CA gene.

The present invention also relates to the method of above, wherein said mutation in the PIK3CA gene is a somatic mutation.

The present invention also relates to any one of the methods above, wherein said mutation in the PIK3CA gene is selected from: 3140A>G, 1633G>A, 1624G>A, 3140A>T, 1634A>C, 1634A>G, 1636C>A, and 333G>C.

The present invention also relates to any one of the methods above, wherein said at least one mutation in the protein encoded by the PIK3CA gene is selected from: H1047L, H1047R, Q546K, E545A, M1043I, E545D, E545K, P539R, K111N, P449T, and E542K.

In one embodiment, said at least one mutation is selected from: H1047R, Q546K, E545A, M1043I, E545D, P539R, and K111N.

In one embodiment, said cancer is selected from: breast, colon, reno cell carcinoma, lung, liver, bladder, melanoma, and lymphatic.

In one embodiment, said at least one first sample is a tumor sample or a tumor cell.

In one embodiment, said human has a tumor with three or more copies of the HER2 gene.

In one embodiment, said human has a tumor with five or more copies of the HER2 gene.

In one embodiment, said human has a tumor that overexpresses Her2 and/or a fragment thereof and/or a protein from a gene encoding Her2.

In one embodiment, said sample does not have a mutation in a KRAS gene.

In one embodiment, said method further comprising determining the RAS protein mutation status from at least one second sample from said human.

In one embodiment, said first sample and said second sample are the same.

In one embodiment, said first sample and said second sample are both tumor samples.

In one embodiment, said first sample and said second sample are from blood.

In one embodiment, said first sample and said second sample are different.

In one embodiment, said Ras protein is KRAS.

In one embodiment, said mutation in said Ras protein is selected from: G12S, G12V, G12D, G12A, G12C, G12R, G13A, G13D, Q61K, Q61R, E76G, E76K, E76Q, and A146T.

In one embodiment, said mutation in said Ras protein is selected from: G12S, G12V, G12D, G12A, G12C, G12R, and G13A.

The present invention also relates to a method of treating a patient with cancer comprising detecting the number of Her2 genes in at least one tumor cell and/or the amount of Her2/neu receptor expressed by said tumor cell from said patient and administering a therapeutically effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition if said tumor cell has 3 or more copies of Her2 gene and/or if said tumor cell expresses a greater amount of a Her 2 gene product than a non-tumor cell.

In one embodiment, said tumor cell is selected from: breast, bladder, pancreatic, lung, colon, melanoma and lymphoid.

The present invention also relates to a method of treating a human with cancer comprising (1) genotyping at least one tumor cell from said human for at least one mutation in a PIK3CA gene, and (2) if at least one mutation in PIK3CA is detected administering at least one dose of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition.

The present invention also relates to a method of treating a human with cancer comprising (1) administering to a human in need thereof an anti-neoplastic agent, (2) genotyping at least one tumor cell from said human for at least one mutation in a PIK3CA gene, and (2) if at least one mutation in PIK3CA is detected administering at least one dose of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition.

In one embodiment, said method further comprising correlating the detection of at least one mutation in PIK3CA with an increased likelihood of response of said human suffering from cancer to 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition.

The present invention also relates to a method of treating a human with cancer comprising (1) genotyping at least one tumor cell from said human for at least one mutation in a PIK3CA gene and for the number of copies of Her2 gene, and (2) if at least one mutation in PIK3CA is detected and at least three copies of Her2 gene is detected, administering at least one dose of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition.

The present invention also relates to any one of the above method, further comprising (1) genotyping at least one tumor cell from said human for at least one mutation in the KRas Protein, (2) if said mutation in Ras protein is not detected, administering at least one dose of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition.

In one embodiment, said at least one mutation of KRas protein is selected from: G12S, G12V, G12D, G12A, G12C, G12R, and G13A.

The present invention also relates to any one of the above method, further comprising administering at least one dose of a second anti-neoplastic agent.

The present invention also relates to a method of treating a human with cancer comprising (1) administering to a human in need thereof a dose of an antineoplastic agent, (2) genotyping at least one tumor cell from said human for at least one mutation in a PIK3CA gene, and (3) if at least one mutation in PIK3CA is detected administering at least one dose of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition.

The present invention also relates to any one of the above methods, further comprising the step of correlating the human's increased likelihood of response to treatment with at least one least one dose of 2,4-difluoro-N-{2-(methyloxy)-5-[4-(4-pyridazinyl)-6-quinolinyl]-3-pyridinyl}benzenesulfonamide or a pharmaceutically acceptable salt if said human has at least one mutation in at least one mutant PI3K protein or a mutation in the PIK3CA gene and/or at least three or more copies of Her2 gene in a tumor cell.

### DEFINITIONS

The term “wild type” as is understood in the art refers to a polypeptide or polynucleotide sequence that occurs in a native population without genetic modification or a state of diploidy for a given genetic locus (2n). A deviation from diploid where a patient has three or more copies of a gene is considered ‘amplified’. As is also understood in the art, a “variant” includes a polypeptide or polynucleotide sequence having at least one modification to an amino acid or nucleic acid compared to the corresponding amino acid or nucleic acid found in a wild type polypeptide or polynucleotide, respectively. Included in the term variant is Single Nucleotide Polymorphism (SNP) where a single base pair distinction exists in the sequence of a nucleic acid strand compared to the most prevalently found (wild type) nucleic acid strand. As used herein “genetic modification” or “genetically modified” refers to, but is not limited to, any suppression, substitution, amplification, deletion and/or insertion of one or more bases into DNA sequence(s). Also, as used herein “genetically modified” can refer to a gene encoding a polypeptide or a polypeptide having at least one deletion, substitution or suppression of a nucleic acid or amino acid, respectively.

Genetic variants and/or SNPs can be identified by known methods. For example, wild type or SNPs can be identified by DNA amplification and sequencing techniques, DNA and RNA detection techniques, including, but not limited to Northern and Southern blot, respectively, and/or various biochip and array technologies. WT and mutant polypeptides can be detected by a variety of techniques including, but not limited to immunodiagnostic techniques such as ELISA and western Blot. DNA amplifications in tumor cells can be identified by quantitative DNA detection techniques such as PCR based methods. In addition, microarray based methods can be used to measure DNA amplifications. These include microarray based comparative genomic hybridization (Greshock, J., et al. 2004. Genome Res 14: 179-87.) and DNA ‘SNP Chips’ (Bignell, G. R., et al. 2004 Genome Res 14: 287-95).

As used herein, the process of detecting an allele or polymorphism includes but is not limited to serologic and genetic methods. The allele or polymorphism detected may be functionally involved in affecting an individual's phenotype, or it may be an allele or polymorphism that is in linkage disequilibrium with a functional polymorphism/allele. Polymorphisms/alleles are evidenced in the genomic DNA of a subject, but may also be detectable from RNA, cDNA or protein sequences transcribed or translated from this region, as will be apparent to one skilled in the art.

As is well known genetics, nucleotide and related amino acid sequences obtained from different sources for the same gene may vary both in the numbering scheme and in the precise sequence. Such differences may be due to numbering schemes, inherent sequence variability within the gene, and/or to sequencing errors. Accordingly, reference herein to a particular polymorphic site by number will be understood by those of skill in the art to include those polymorphic sites that correspond in sequence and location within the gene, even where different numbering/nomenclature schemes are used to describe them.

As used herein, “genotyping” a subject (or DNA or other biological sample) for a polymorphic allele of a gene(s) or a mutation in at least one polypeptide or gene encoding at least one polypeptide means detecting which mutated, allelic or polymorphic form(s) of the gene(s) or gene expression products (e.g., hnRNA, mRNA or protein) are present or absent in a subject (or a sample). Related RNA or protein expressed from such gene may also be used to detect mutant or polymorphic variation. As is well known in the art, an individual may be heterozygous or homozygous for a particular allele. More than two allelic forms may exist, thus there may be more than three possible genotypes. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g., sequencing) or indirectly (e.g., by restriction fragment length polymorphism analysis, or detection of the hybridization of a probe of known sequence, or reference strand conformation polymorphism), or by using other known methods.

As used herein, a “genetic subset” of a population consists of those members of the population having a particular genotype or a tumor having at least one somatic mutation. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria, e.g., individuals being treated with Compound B or individuals with cancer. In some instances, a genetic subset of a population may have a higher likelihood of response to treatment compared with another genetic subset. For instance, a genetic subset of cancer patients with an amplification of the HER2 gene may have a greater percentage of response to treatment with Compound B than a subset without that amplification. By way of another example, patients with a particular genotype may demonstrate an increased risk or decreased risk of a particular phenotypic response.

As used herein, a subject that is “predisposed to” or “at increased risk of” a particular phenotypic response based on genotyping will be more likely to display that phenotype than an individual with a different genotype at the target polymorphic locus (or loci). Where the phenotypic response is based on a multi-allelic polymorphism, or on the genotyping of more than one gene, the relative risk may differ among the multiple possible genotypes.

As used herein “response” to treatment and grammatical variations thereof, includes but is not limited to an improved clinical condition of a patient after the patient received medication. Response can also mean that a patient's condition does not worsen upon that start of treatment. Response can be defined by the measurement of certain manifestations of a disease or disorder. With respect to cancer, response can mean, but is not limited to, a reduction of the size and or number of tumors and/or tumor cells in a patient. Response can also be defined by a other endpoints such as a reduction or attenuation in the number of pre-tumorous cells in a patient.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

Biological samples for testing of one or more mutations may be selected from the group of proteins, nucleotides, cellular blebs or components, serum, cells, blood, blood components such as circulating tumor DNA, urine and saliva. Testing for mutations may be conducted by several techniques known in the art and/or described herein.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations, polymorphisms, and cytogenetic alterations (e.g. DNA amplifications and deletions) (Teton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996); (Greshock, J., et al. 2004. Genome Res 14: 179-87; Bignell, G. R., et al. 2004 Genome Res 14: 287-95).). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The terms “polypeptide” and “protein” are used interchangeably and are used herein as a generic term to refer to native protein, fragments, peptides, or analogs of a polypeptide sequence. Hence, native protein, fragments, and analogs are species of the polypeptide genus.

The terminology “X#Y” in the context of a mutation in a polypeptide sequence is art-recognized, where “#” indicates the location of the mutation in terms of the amino acid number of the polypeptide, “X” indicates the amino acid found at that position in the wild-type amino acid sequence, and “Y” indicates the mutant amino acid at that position. For example, the notation “G125” with reference to the K-ras polypeptide indicates that there is a glycine at amino acid number 12 of the wild-type K-ras sequence, and that glycine is replaced with a serine in the mutant K-ras sequence.

The term “at least one mutation” in a polypeptide or a gene encoding a polypeptide and grammatical variations thereof means a polypeptide or gene encoding a polypeptide having one or more allelic variants, splice variants, derivative variants, substitution variants, deletion variants, and/or insertion variants, fusion polypeptides, orthologs, and/or interspecies homologs. By way of example, at least one mutation of PIK3CA would include a PIK3CA in which part of all of the sequence of a polypeptide or gene encoding the polypeptide is absent or not expressed in the cell for at least one of the PIK3CA proteins produced in the cell. For example, a PIK3CA protein may be produced by a cell in a truncated form and the sequence of the truncated form may be wild type over the sequence of the truncate. A deletion may mean the absence of all or part of a gene or protein encoded by a gene. Additionally, some of a protein expressed in or encoded by a cell may be mutated while other copies of the same protein produced in the same cell may be wild type.

As used herein “genetic abnormality” is meant a deletion, substitution, addition, translocation, amplification and the like relative to the normal native nucleic acid content of a cell of a subject. The terms “mutant PIK3CA” and “PIK3CA mutant” refer to PIK3 proteins having at least one mutation. In certain embodiments, the mutations include but are not limited to, mutations at amino acids H 1407, E545, P539, P449 and E542, including but not limited to, H1407L, H1407R, E545K, P539R, P449T, and E542K. Certain exemplary mutant PIK3CA polypeptides include, but are not limited to, allelic variants, splice variants, derivative variants, substitution variants, deletion variants, and/or insertion variants, fusion polypeptides, orthologs, and interspecies homologs. In certain embodiments, a mutant PIK3CA polypeptides includes additional residues at the C- or N-terminus, such as, but not limited to, leader sequence residues, targeting residues, amino terminal methionine residues, lysine residues, tag residues and/or fusion protein residues.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes, although oligonucleotides may be double stranded, e.g. for use in the construction of a gene mutant. Oligonucleotides can be either sense or antisense oligonucleotides.

An oligonucleotide probe, or probe, is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al., supra or Glick et al., supra).

The term “amplification” and grammatical variations thereof refers to the presence of one or more extra gene copies in a chromosome complement. As used herein a HER2 gene maybe amplified if 3 or more copies of the gene exist in the cell. Similarly, amplification would also include 3, 4, 5, 6 or more copies of a gene in a cell. Amplification of the HER2 gene has been found in to be frequent in breast cancers, and has been noted to occur in other tumor types such as stomach cancers Semba et al., Proc. Natl. Acad. Sci. USA, 82:6497-6501 (1985); Yokota et al., Oncogene, 2:283-287 (1988); Zhou et al., Cancer Res., 47:6123-6125 (1987); King et al., Science, 229:974-976 (1985); Kraus et al., EMBO J., 6:605-610 (1987); van de Vijver et al., Mol. Cell. Biol., 7:2019-2023 (1987); Yamamoto et al., Nature, 319:230-234 (1986).

The terms “HER2 amplified” and “amplified HER2” refer to a state where cells have greater than normal (2 copies) of the HER2 locus which maps to 17q21-q22. The amplification of HER2 can also encompass neighboring genes (e.g. GRB7). Also, amplifications can be of different magnitudes, such as cells with 3 copies as well as those with >20 copies.

As used herein “overexpressed” and “overexpression” and grammatical variations thereof means that a given cell produces an increased number of a certain protein relative to a normal cell. For instance, some tumor cells are known to overexpress Her2 or Erb2 on the cell surface compared with cells from normal breast tissue. Gene transfer experiments have shown that overexpression of HER2 will transform NIH 3T3 cells and also cause an increase in resistance to the toxic macrophage cytokine tumor necrosis factor. Hudziak et al., “Amplified Expression of the HER2/ERBB2 Oncogene Induces Resistance to Tumor Necrosis Factor Alpha in NIH 3T3 Cells”, Proc. Natl. Acad. Sci. USA 85, 5102-5106 (1988). Expression levels of a polypeptide in a particular cell can be effected by, but not limited to, mutations, deletions and/or substitutions of various regulatory elements and/or non-encoding sequence in the cell genome.

As used herein, “treatment” means any manner in which one or more symptoms associated with the disorder are beneficially altered. Accordingly, the term includes healing or amelioration of a symptom or side effect of the disorder or a decrease in the rate of advancement of the disorder.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. Tumors may be hematopoietic tumor, for example, tumors of blood cells or the like. Specific examples of clinical conditions based on such a tumor include leukemia such as chronic myelocytic leukemia or acute myelocytic leukemia; myeloma such as multiple myeloma; lymphoma and the like.

In some embodiments, the biological sample is selected from the group consisting of cells, including tumor cells, blood, blood components, urine and saliva.

In some embodiments, the biological sample is selected from the group consisting of tumor cells, cells, blood, blood components, urine and saliva.

While it is possible that Compound B, as well as pharmaceutically acceptable salts and solvates thereof, may be administered as the raw chemical, it is also possible to present the active ingredient as a pharmaceutical composition. Accordingly, embodiments of the invention further provide pharmaceutical compositions, which include therapeutically effective amounts of Compound B, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing Compound B with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 800 mg, of a compound of the Compound B depending on the condition being treated, the route of administration and the age, weight and condition of the patient. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

Dosage unit forms can also be in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

It should be understood that in addition to the ingredients particularly mentioned above, the formulations may include other agents conventional in the art having regard to the type of formulation in question, for example those suitable for oral administration may include flavouring agents.

A therapeutically effective amount of Compound B or a pharmaceutically acceptable salt or solvate thereof will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of Compound B or a salt or solvate thereof for the treatment of a cancerous condition such as those described herein will generally be in the range of 0.1 to 100 mg/kg body weight of recipient (mammal) per day and more usually in the range of 1 to 12 mg/kg body weight per day. Thus, for a 70 kg adult mammal, the actual amount per day would usually be from 70 to 840 mg and this amount may be given in a single dose per day or more usually in a number (such as two, three, four, five or six) of sub-doses per day such that the total daily dose is the same. An effective amount of a salt or solvate thereof may be determined as a proportion of the effective amount of Compound B per se. It is envisaged that similar dosages would be appropriate for treatment of the other conditions referred to above.

The amount of administered or prescribed compound according to these aspects of the present invention will depend upon a number of factors including, for example, the age and weight of the patient, the precise condition requiring treatment, the severity of the condition, the nature of the formulation, and the route of administration. Ultimately, the amount will be at the discretion of the attendant physician.

Response Evaluation Criteria In Solid Tumors (RECIST) is a set of published rules that define when cancer patients improve (“respond”), stay the same (“stabilize”), or worsen (“progression”) during treatments. The criteria were published in February, 2000 by an international collaboration including the European Organisation for Research and Treatment of Cancer (EORTC), National Cancer Institute of the United States, and the National Cancer Institute of Canada Clinical Trials Group. Today, the majority of clinical trials evaluating cancer treatments for objective response in solid tumors are using RECIST.

### RECIST 1.0 Criteria

### Definition of Measurable and Non-Measurable Disease

Measurable disease: The presence of at least one measurable lesion.
Measurable lesion: Lesions that can be accurately measured in at least one dimension, with the longest diameter (LD) being:
≧20 mm with conventional techniques (medical photograph [skin or oral lesion], palpation, plain X-ray, CT, or MRI),
OR
≧10 mm with spiral CT scan.

Non-measurable lesion: All other lesions including lesions too small to be considered measurable (longest diameter <20 mm with conventional techniques or <10 mm with spiral CT scan) including bone lesions, leptomeningeal disease, ascites, pleural or pericardial effusions, lymphangitis cutis/pulmonis, abdominal masses not confirmed and followed by imaging techniques, cystic lesions, or disease documented by indirect evidence only (e.g., by lab values).

### Methods of Measurement

Conventional CT and MRI: Minimum sized lesion should be twice the reconstruction interval. The minimum size of a baseline lesion may be 20 mm, provided the images are reconstructed contiguously at a minimum of 10 mm. MRI is preferred, and when used, lesions must be measured in the same anatomic plane by use of the same imaging sequences on subsequent examinations. Whenever possible, the same scanner should be used.
Spiral CT: Minimum size of a baseline lesion may be 10 mm, provided the images are reconstructed contiguously at 5 mm intervals. This specification applies to the tumors of the chest, abdomen, and pelvis.
Chest X-ray: Lesions on chest X-ray are acceptable as measurable lesions when they are clearly defined and surrounded by aerated lung. However, MRI is preferable.
Clinical Examination: Clinically detected lesions will only be considered measurable by RECIST criteria when they are superficial (e.g., skin nodules and palpable lymph nodes). In the case of skin lesions, documentation by color photography—including a ruler and patient study number in the field of view to estimate the size of the lesion—is required.

### Baseline Documentation of Target and Non-Target Lesions

All measurable lesions up to a maximum of five lesions per organ and ten lesions in total, representative of all involved organs, should be identified as target lesions and recorded and measured at baseline.

Target lesions should be selected on the basis of their size (lesions with the LD) and their suitability for accurate repeated measurements (either clinically or by imaging techniques).

A sum of the LD for all target lesions will be calculated and reported as the baseline sum LD. The baseline sum LD will be used as a reference by which to characterize the objective tumor response.

All other lesions (or sites of disease) should be identified as non-target lesions and should also be recorded at baseline. Measurements of these lesions are not required, but the presence or absence of each should be noted throughout follow-up.

Documentation of indicator lesion(s) should include date of assessment, description of lesion site, dimensions, and type of diagnostic study used to follow lesion(s).

All measurements should be taken and recorded in metric notation, using a ruler or callipers.

### Response Criteria

Disease assessments are to be performed every 6 weeks after initiating treatment. However, subjects experiencing a partial or complete response must have a confirmatory disease assessment at least 28 days later. Assessment should be performed as close to 28 days later (as scheduling allows), but no earlier than 28 days.

Definitions for assessment of response for target lesion(s) are as follows:

### Evaluation of Target Lesions

Complete Response (CR)—disappearance of all target lesions.
Partial Response (PR)—at least a 30% decrease in the sum of the LD of target lesions, taking as a reference, the baseline sum LD.
Stable Disease (SD)—neither sufficient shrinkage to qualify for PR nor sufficient increase to qualify for progressive disease (PD), taking as a reference, the smallest sum LD since the treatment started. Lesions, taking as a reference, the smallest sum LD recorded since the treatment started or the appearance of one or more new lesions.

### Evaluation of Non-Target Lesions

Definitions of the criteria used to determine the objective tumor response for non-target lesions are as follows:

Complete Response—the disappearance of all non-target lesions.
Incomplete Response/Stable Disease—the persistence of one or more non-target lesion(s).
Progressive Disease—the appearance of one or more new lesions and/or unequivocal progression of existing non-target lesions.

### Evaluation of Overall Response for RECIST-Based Response

The overall response is the best response recorded from the start of the treatment until disease progression/recurrence is documented. In general, the subject's best response assignment will depend on the achievement of both measurement and confirmation criteria.

The following table presents the evaluation of best overall response for all possible combinations of tumor responses in target and non-target lesions with or without the appearance of new lesions.

| Target Lesion | Non-Target Lesion | New Lesion | Overall response |
|---|---|---|---|
| CR | CR | No | CR |
| CR | Incomplete | No | PR |
| | response/(SD) | | |
| PR | Non-PD | No | PR |
| SD | Non-PD | No | SD |
| PD | Any | Yes or No | PD |
| Any | PD | Yes of No | PD |
| Any | Any | Yes | PD |

Note: Subjects with a global deterioration of health status requiring discontinuation of treatment without objective evidence of disease progression at that time should be classified as having “symptomatic deterioration”. Every effort should be made to document the objective progression even after discontinuation of treatment.

In some circumstances, it may be difficult to distinguish residual disease from normal tissue. When the evaluation of complete response depends on this determination, it is recommended that the residual lesion be investigated (fine needle aspirate/biopsy) to confirm the complete response status.

### Confirmation Criteria

To be assigned a status of PR or CR, a confirmatory disease assessment should be performed no less than 28 days after the criteria for response are first met.

To be assigned a status of SD, follow-up measurements must have met the SD criteria at least once after study entry at a minimum interval of 12 weeks.

### EXPERIMENTALS

### Preparation of Compound B

### 2,4-difluoro-N-{2-(methyloxy)-5-[4-(4-pyridazinyl)-6-quinolinyl]-3-pyridinyl}benzenesulfonamide

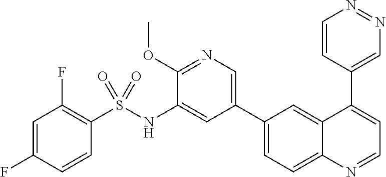 [SMILES: COc1ncc(-c2ccc3nccc(-c4ccnnc4)c3c2)cc1NS(=O)(=O)c1ccc(F)cc1F]

### a) 6-bromo-4-(4-pyridazinyl)quinoline

Dissolved 6-bromo-4-iodoquinoline (17.43 g, 52.2 mmol), 4-(tributylstannanyl)pyridazine (19.27 g, 52.2 mmol), and PdCl2(dppf)-CH₂Cl₂ (2.132 g, 2.61 mmol) in 1,4-dioxane (200 mL) and heated to 105° C. After 3 h, added more palladium catalyst and heated for 6 h. Concentrated and dissolved in methylene chloride/methanol. Purified by column chromatography (combiflash) with 2% MeOH/EtOAc to 5% MeOH/EtOAc to give the crude title compound. Trituration with EtOAc furnished 6-bromo-4-(4-pyridazinyl)quinoline (5.8 g, 20.27 mmol, 38.8% yield). MS(ES)+ m/e 285.9, 287.9 [M+H]⁺.

### b) 2,4-difluoro-N-{2-(methyloxy)-5-[4-(4-pyridazinyl)-6-quinolinyl]-3-pyridinyl}benzenesulfonamide

A slurry of 6-bromo-4-(4-pyridazinyl)quinoline (4.8 g, 16.78 mmol), bis(pinacolato)diboron (4.69 g, 18.45 mmol), PdCl2(dppf)-CH₂Cl₂ (530 mg, 0.649 mmol) and potassium acetate (3.29 g, 33.6 mmol) in anhydrous 1,4-dioxane (120 ml) was heated at 100° C. for 3 h. The complete disappearance of the starting bromide was observed by LCMS. The reaction was then treated with N[5-bromo-2-(methyloxy)-3-pyridinyl]-2,4-difluorobenzenesulfonamide (6.68 g, 17.61 mmol) and another portion of PdCl2(dppf)-CH₂Cl₂ (550 mg, 0.673 mmol), then heated at 110° C. for 16 h. The reaction was allowed to cool to room temperature, filtered, and concentrated. Purification of the residue by chromatography (Analogix; 5% MeOH/5% CH2Cl2/90% EtOAC) gave 6.5 g (76%) desired product. MS(ES)+ m/e 505.9 [M+H]⁺.

### Intermediates

### Preparation of N-[5-bromo-2-(methyloxy)-3-pyridinyl]-2,4-difluorobenzenesulfonamide

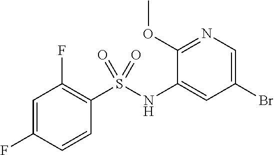 [SMILES: COc1ncc(Br)cc1NS(=O)(=O)c1ccc(F)cc1F]

### a) 5-bromo-2-(methyloxy)-3-nitropyridine

To a cooled (0° C.) solution of 5-bromo-2-chloro-3-nitropyridine (50 g, 211 mmol) in methanol (200 mL) was added dropwise over 10 minutes 20% sodium methoxide (50 mL, 211 mmol) solution. The reaction, which quickly became heterogeneous, was allowed to warm to ambient temperature and stirred for 16 h. The reaction was filtered and the precipitate diluted with water (200 mL) and stirred for 1 h. The solids were filtered, washed with water (3×100 mL) and dried in a vac oven (40° C.) to give 5-bromo-2-(methyloxy)-3-nitropyridine (36 g, 154 mmol, 73.4% yield) as a pale yellow powder. The original filtrate was concentrated in vacuo and diluted with water (150 mL). Saturated ammonium chloride (25 mL) was added and the mixture stirred for 1 h. The solids were filtered, washed with water, and dried in a vac oven (40° C.) to give a second crop of 5-bromo-2-(methyloxy)-3-nitropyridine (9 g, 38.6 mmol, 18.34% yield). Total yield=90%. MS(ES)+m/e 232.8, 234.7 [M+H]⁺.

### b) 5-bromo-2-(methyloxy)-3-pyridinamine

To a solution of 5-bromo-2-(methyloxy)-3-nitropyridine (45 g, 193 mmol) in ethyl acetate (1 L) was added tin(II) chloride dihydrate (174 g, 772 mmol). The reaction mixture was heated at reflux for 4 h. LC/MS indicated some starting material remained, so added 20 mol % tin (II) chloride dihydrate and continued to heat at reflux. After 2 h, the reaction was allowed to cool to ambient temperature and concentrated in vacuo. The residue was treated with 2 N sodium hydroxide and the mixture stirred for 1 h. The mixture was then with methylene chloride (1 L), filtered through Celite, and washed with methylene chloride (500 mL). The layers were separated and the organics dried over magnesium sulfate and concentrated to give 5-bromo-2-(methyloxy)-3-pyridinamine (23 g, 113 mmol, 58.7% yield). The product was used crude in subsequent reactions. MS(ES)+ m/e 201.9, 203.9 [M+H]⁺.

### c) N-[5-bromo-2-(methyloxy)-3-pyridinyl]-2,4-difluorobenzenesulfonamide

To a cooled (0° C.) solution of 5-bromo-2-(methyloxy)-3-pyridinamine (20.3 g, 100 mmol) in pyridine (200 mL) was added slowly 2,4-difluorobenzenesulfonyl chloride (21.3 g, 100 mmol) over 15 min (reaction became heterogeneous). The ice bath was removed and the reaction was stirred at ambient temperature for 16 h, at which time the reaction was diluted with water (500 mL) and the solids filtered off and washed with copious amounts of water. The precipitate was dried in a vacuum oven at 50° C. to give N-[5-bromo-2-(methyloxy)-3-pyridinyl]-2,4-difluorobenzenesulfonamide (12 g, 31.6 mmol, 31.7% yield) MS(ES)+ m/e 379.0, 380.9 [M+H]⁺.

### Biological Data:

### Genotyping

DNA was extracted from blood using the Qiagen QiAmp DNA Blood Kit. Genotyping was conducted using the following technologies: Illumine Human 1M DNA Analysis Beadchip platform (Steemers F J, Chang W, Lee G, Barker D L, Shen R et al. (2006) Whole-genome genotyping with the single-base extension assay. Nat Methods 3: 31-33) a single base chain extension assay modified by GlaxoSmithKline (Taylor J D, Briley D, Nguyen Q, Long K, Iannone M A, Li M S, Ye F, Afshari A, Lai E, Wagner M, Chen J, Weiner M P (2001) Flow cytometric platform for high-throughput single nucleotide polymorphism analysis. Biotechniques 30(3): 661-6, 668-9).

### Statistical Analysis

Efficacy PGx analyses were conducted for each polymorphism using PFS and response rate (RECIST) based on Investigator Review as endpoints. Cox regression was used to investigate genetic association of each SNP with PFS. Kaplan-Meier plots of survival by genotype were produced. Each of the following covariates—age, sex, race, Motzer risk score, ECOG performance status, and prior nephrectomy status—were individually tested for association with PFS by Cox modeling. All covariates that are significantly associated with PFS at p<0.05 were included in the Cox model for genotype.

RECIST responses were grouped into 3 categories: partial and complete responders (PR+CR), stable disease (SD), and progressive disease (PD). Patients with “unknown” or “not evaluable” responses status were excluded in this analysis. Fisher's exact test of proportions was used on the 3×3 table formed between response and genotype to assess the significance of the association.

No adjustments for multiple comparisons were made in this exploratory analysis. Polymorphisms that met nominal levels of significance (p<0.05) were reported which will optimally require confirmation in an independent dataset.

### Example 1

The phosphoinositide 3-kinase (PI3K) signalling pathway is activated in a broad spectrum of human cancers. The biological role of PI3K in growth and survival of cancer cells and the prevalence of activating mutations in human cancers are well documented. Activation of this pathway often occurs indirectly by somatic aberrations of receptor tyrosine kinases, or directly by mutations in PI3K genes, such as PIK3CA. A significant proportion of tumors would be predicted to benefit from inhibition of this pathway.

Compound B also referred to as 2,4-difluoro-N-{2-(methyloxy)-5-[4-(4-pyridazinyl)-6-quinolinyl]-3-pyridinyl}benzenesulfonamide is a novel, orally administered inhibitor of wild type phosphoinositide 3-kinase alpha (PI3Kα) and the common activation mutants of p110α found in human cancers. Compound B demonstrates good selectivity for the PI3K family of enzymes when evaluated in a large panel of protein kinases. This compound has has recently entered Phase I clinical trials.

As efforts to predict clinical efficacy using cohorts of in vitro tumor models have been successful, expansive panels of tumor derived cell lines can recapitulate an ‘all comers’ efficacy trial; thereby identifying which histologies and specific tumor genotypes are most likely to benefit from treatment. To this end, Compound B was tested against two a panels of human breast tumor cell lines.

### Study 1. Breast Cancer Cell Line Panel A.

### Methods

### Cell Line Proliferation Assays

A total of 15 breast cancer cell lines were used in this study. These cells were cultured in RPMI-1640 and supplemented with 5% or 10% fetal bovine serum, 2 mM GlutaMAX™ and 1 mM sodium pyruvate, or in growth medium recommended by the suppliers [American Type Culture Collection, Manassas, Va., USA; Developmental Therapeutics Program, National Cancer Institute, Bethesda, Md., USA; German Collection of Microorganisms and Cell Cultures (DSMZ), Braunschweig, Germany; European Collection of Cell Cultures (ECACC), Porton Down, UK]. Cells of each line were seeded into a 384-well microtiter plate at two cell densities. Low density ranges were 300-1800 cells/well, and a high density was 600 to 3600 cells/well). The plating density was determined by the rate of proliferation of the cell line in the absence of any inhibitor and varied amongst cell lines. High plating densities were double that of low density. Each cell density was plated in triplicate. In total there were 30 wells to be treated with increasing concentrations of Compound B for each cell line at each density. In addition, there were 24 wells of dimethyl sulfoxide (DMSO)-treated controls at each density. After seeding, the cells were incubated at 37° C. in 5% CO₂ for 24 hours. Subsequently, Compound B was added to each cell line with 10 concentrations. The dosing solution of Compound B was first prepared in DMSO at a master stock concentration of 10 mM. 1:3 serial dilutions were then made of the stock solution to give a range of working stock concentrations; 10.00 mM, 3.164 mM, 1.001 mM, 316.9 μM, 100.3 μM, 31.74 μM, 10.04 μM, 3.178 μM, 1.006 μM and 318.3 nM. The working stock concentrations of Compound B were then dispensed into the seeded cancer cell lines using a Biomek FX liquid handler to give final treatment concentrations of 0.1% of the stock concentration. A similar volume of DMSO without Compound B was dispensed into the 24 control wells of each seeding density at a concentration of 0.1%. Also, a zero-time plate prepared with a similar cell seeding was read for each cell line at each seeding density immediately after the addition of the DMSO control. After a 72 h incubation, an equal volume of the CellTiter-Glo (CellTiter-Glo Luminescent Cell Viability Assay, Promega, Madison, Wis.) to that of the cell culture medium was added into each well of the plate. After the contents were mixed to induce cell lysis and stabilization, cell luminescence was recorded using a SpectraMax M5^{e} (Molecular Devices, Sunnyvale, Calif., USA)

The luminescence of Compound B treated cells was compared relative to the average of the 24 DMSO-treated control wells at each cell density for every concentration of Compound B for all triplicate wells. The percent intensity values were used in model 205 of XLfit in Microsoft Excel to calculate gIC₅₀s for the low and high seeding density (a 4 parameter logistical fit). Specifically, the midpoint of the growth window (the gIC₅₀) falls half way between the number of cells at the time of compound addition (T=0) and the growth of control cells treated with DMSO. The gIC₅₀ value serves as a metric for measuring the inhibition of proliferation in cancer cells. The curves for each seeding density for each cell line were manually inspected for both data quality and appropriateness of curve fitting. Where the starting data were poor the curve and subsequent gIC₅₀s were excluded from further analysis. Cell line seedings were considered of poor quality when the r² value of the fitted curve was <0.75. Finally, where data quality permits, the Ymin values (defined as the maximum growth inhibition relative to the cell density at Time-zero) were calculated as a measure of cytotoxicity where data were of reasonable quality.

DNA Copy number data on the HER2 gene was collected for all 15 cell lines using the Affymetrix 500K chip (Affymetrix Inc, Sunnyvale, Calif.). First, DNA was extracted from each line using GenElute Mammalian Genomic DNA miniprep kit (Sigma, St. Louis, Mo.). Two aliquots (250 ng each) were digested with the restriction enzyme Nsp or Sty (New England Biolabs, Boston, Mass.). Digested DNA was subsequently ligated to an adaptor and amplified by PCR using Platinum Pfx DNA Polymerase (Invitrogen), yielding a product of approximately 250-2000 bp. For each enzyme digest, PCR was carried out in four 100 μL aliquots, pooled, purified, quantified, normalized to 40 μg/45 μL and fragmented with DNase I to yield a size range of approximately 25-200 bp. The fragmented products of the cancer cell lines were then labeled, denatured, and hybridized to the Affymetrix 500K chip. Upon completion of hybridization, each assay was washed and stained using Affymetrix fluidics stations. Image data were acquired using the GeneChip Scanner 3000 (Expression Analyisis, Inc, Durham N.C.). Similarly collected data from a panel 10 non-tumorigenic lymphoblastic cell lines were used to calculate DNA copy number.

DNA copy number for the HER2 gene was calculated using the following procedures:
1. All ‘SNP Chip’ images (‘CEL files’), were extracted using the Affymetrix Genotype software, and read and normalized using the dChip software package (Lin et al. 2004). A SNP-wise ‘copy-number ratios’ (log₂ scale) were calculated for all cancer cell lines by dividing the SNP intensity score by the respective median intensity score for the lymphoblastic reference panel. Data were adjusted under the assumption that the median copy number for all samples was diploid.
2. Finally, copy number inferences were made by circular binary segmentation (CBS) to reduce noise (e.g. from unmasked complex sequences in the target) and provide a consensus score for all regions of the genome based on at least two underlying SNP (Olshen et al. 2004).
3. Log₂ ratio cutoffs based upon previous comparisons of this platform to karyotype data (Greshock, J., et al. 2007. Cancer Res 67:10173-80) were used to classify HER2 as having copy number gains of 3-5 copies (0.25-0.65), gains >5 copies (>0.65), monsomies (−0.25-−0.75) or homozygous losses (<−0.75).

### Cell Line Mutation Data

Mutation data was collated for the status for the PIK3CA and KRAS gene. The data source is the cancer cell line mutation screening data published as part of the Catolog of Somatic Mutations in Cancer database (COSMIC) (Bamford S. et al. Br. J. Cancer. 2004. 91:355-58). In order to ensure that the identity of the cell lines used in the proliferation assay matched that in the COSMIC database, a genotype comparison was done between those cell lines in the sensitivity screen and those in COSMIC. Specifically, this entailed:
1. Calculating the genotypes for each cell line using the Affymetrix 500K ‘SNP Chip’(Affymetrix, Inc., Sunnyvale, Calif.) and the RLMM algorithm (Rabbee & Speed, Bioinformatics, 2006. 22: 7-12).
2. Identifying the genotype matches of each cell line to those pre-calculated for each cell line having mutation profiles in COSMIC.
   Assigning mutation status for each cell line in based upon the genotype matches.

### Results

Compound B was tested in a panel of 15 human breast cell lines. Cytotoxicity curves were generated and gIC₅₀s determined for all cells using two cell densities (Table 1). gIC₅₀s for Compound B across the 15 cell panel ranged from 0.1 to 227.0 nM. The overall median gIC₅₀ was 3.2 nM. Only 3/15 (20%) tumor cell lines demonstrated a gIC₅₀>20 nM, while 7/15 (47%) had gIC₅₀s<3 nM.

The degree of responsiveness for each individual cell line was measured based upon gIC₅₀ calculations where lower values are more responsive the cell was to treatment with Compound B.

Mutation data for KRAS and PIK3CA was available for all 15 cell lines screened for responsiveness to Compound B. A total of 40% ( 6/15) cell lines had mutations of PIK3CA, and 7% ( 1/15) had mutations of KRAS. No cell line had mutations to both genes. A total of 20% ( 3/15) had copy number gains of 5 copies of the HER2 gene. These data are presented in Table 2.

### DNA Sequences:

Wild Type gene sequence for human PIK3CA is known in the art and available through various databases including: http://www.ncbi.nlm.nih.gov/, with a NCBI Reference Sequence: NG_{—}012113.1. See Also Volinia, et al. Genomics 24(3):472-7 (1994).

KRas: gene sequence is also available though NCBI database, http://www.ncbi.nlm.nih.gov, NCBI Reference Sequence: NG_{—}007524.1

The wild type protein sequences for K-Ras, N-Ras, and H-Ras are known in the art and can be obtained from various databases including SwisProt database UniProtKB/Swiss-Prot: UniProtKB No. P01116 (K-ras); UniProtKB No. P01111 (N-ras), and P01112 (H-Ras), respectively. Also see Shimizu, et al., Proc. Natl. Acad. Sci. (U.S.A.), 80 (1983), pp. 2112-2116; Bos, Mutation research, Reviews in Genetic Toxicology 195 (30:255-271 (1988); and Fasano, et al., Mol. Cell. Biol., 4 (1984), pp. 1695-1705.

### Mutations:

PIK3CA

| DNA Change | Protein Change |
|---|---|
| 3140A > G | H1047R |
| 1633G > A | Q546K |
| 1624G > A | E545A |
| 3140A > T | M1043I |
| 1634A > C | E545D |
| 1634A > G | E545D |
| 1636C > A | P539R |
| 333G > C | K111N |

KRAS

| DNA Change | Protein Change |
|---|---|
| 38G > A | G13D |
| 34G > T | G12C |
| 35G > A | G12D |
| 35G > T | G12V |
| 34G > A | G12S |
| 34G > C | G12R |
| 35G > C | G12A |

TABLE 1
Activity of Compound B in 15 human breast cancer cell lines

| | | | Low | High | Mean | Low | High |
|---|---|---|---|---|---|---|---|
| | | | Density | Density | gIC₅₀ | Density | Density Y- |
| Cell Line | Site/Type | Dx/Histology | gIC₅₀ (nM) | gIC₅₀ (nM) | (nM) | Y-Min | Min |
| EFM-19 | Breast | carcinoma | 0.6 | 0.5 | 0.55 | 24.1 | 22.7 |
| ZR-75-1 | Breast | carcinoma | 0.8 | 0.9 | 0.85 | 19.8 | 22.1 |
| MDA-MB-175-VII | Breast | carcinoma | 1.7 | 1.1 | 1.4 | 19.8 | 12.2 |
| T-47D | Breast | carcinoma | 1.1 | 1.8 | 1.45 | 104.6 | 103.6 |
| MCF7 | Breast | adenocarcinoma | 2.5 | 1.1 | 1.8 | 78.5 | 82.2 |
| KPL-1 | Breast | carcinoma | 2 | 2.7 | 2.35 | 126.6 | 130.2 |
| HCC1954 | Breast | carcinoma | 3 | 3.4 | 3.2 | 19.8 | 18.3 |
| SK-BR-3 | Breast | adenocarcinoma | 2.9 | 4.2 | 3.55 | 52.3 | 54.5 |
| HCC70 | Breast | carcinoma | 4.6 | 4.8 | 4.7 | 9.6 | 8.7 |
| BT-20 | Breast | carcinoma | 6.3 | 7.8 | 7.05 | 10.7 | 20.7 |
| DU4475 | Breast | carcinoma | 3.6 | 20.6 | 12.1 | 1.2 | −8.4 |
| MDA-MB-468 | Breast | carcinoma | 37.2 | 32.1 | 34.9 | 27.9 | 35.5 |
| NCI/ADR-RES | Breast | Carcinoma | 51.6 | 25.4 | 38.5 | 518.6 | 98.9 |
| MDA-MB-231 | Breast | carcinoma | 97.1 | 357.0 | 227.0 | 189.1 | 357.2 |
| UACC-812 | Breast | carcinoma | 0.1 | 0.1 | 0.1 | 12.1 | 12.6 |

Cell Line = Tumor-derived cell line
Site/Type = Site of malignancy or tumor type
DX/Histology = Diagnosis of cancer or histological subtype
gIC₅₀ = Concentration of compound required to cause 50% growth inhibition
Y-min = The minimum cellular growth in the presence of Compound B (relative to DMSO control) as measured by % of that at T = 0 (number of cells at time of Compound B addition). A negative number indicates a net loss of cells relative to that at T = 0.

TABLE 2
Mutations and DNA copy number changes noted in 15 breast cancer cell lines

| | | | Mean | | | |
|---|---|---|---|---|---|---|
| | | | gIC₅₀ | | | |
| Cell Line | Site/Type | DX/Histology | (nM) | Her2 Copies | KRAS | PIK3CA |
| EFM-19 | Breast | carcinoma | 0.55 | Gain <5 | WT | p.H1047L |
| ZR-75-1 | Breast | carcinoma | 0.85 | 2 copies | WT | WT |
| MDA-MB- | Breast | carcinoma | 1.4 | 2 copies | WT | WT |
| 175-VII | | | | | | |
| T-47D | Breast | carcinoma | 1.45 | Gain <5 | WT | p.H1047R |
| MCF7 | Breast | adenocarcinoma | 1.8 | Loss 1 copy | WT | p.E545K |
| KPL-1 | Breast | carcinoma | 2.35 | 2 copies | WT | p.E545K |
| HCC1954 | Breast | carcinoma | 3.2 | Gain ≧5 | WT | p.H1047R |
| SK-BR-3 | Breast | adenocarcinoma | 3.55 | Gain ≧5 | WT | WT |
| HCC70 | Breast | carcinoma | 4.7 | Loss 1 copy | WT | WT |
| BT-20 | Breast | carcinoma | 7.05 | 2 copies | WT | p.P539R |
| DU4475 | Breast | carcinoma | 12.1 | 2 copies | WT | WT |
| MDA-MB- | Breast | carcinoma | 34.9 | 2 copies | WT | WT |
| 468 | | | | | | |
| NCI/ADR- | Breast | carcinoma | 38.5 | 2 copies | WT | WT |
| RES | | | | | | |
| MDA-MB- | Breast | carcinoma | 227.0 | 2 copies | p.G13D | WT |
| 231 | | | | | | |
| UACC-812 | Breast | carcinoma | 0.1 | Gain ≧5 | WT | WT |

Table 2 Key:
Cell Line = Tumor-derived cell line
Site/Type = Site of malignancy or tumor type
DX/Histology = Diagnosis of cancer or histological subtype
Mean gIC₅₀ = Concentration of compound required to cause 50% growth inhibition
HER2 Copies = Estimation of the number of copies of the HER2 gene.
KRAS/PIK3CA = WT = Wild Type

### Study 2. Breast Cell Line Panel B

Proliferation inhibition as a function of Compound B treatment was analyzed in a separate assay in a panel of 51 breast cell lines composed of both normal epithelial tissues and cancer cells

### Drug Preparation:

Drugs were dissolved in DMSO as a 33 mM (unless otherwise stated) stock and stored at −20 C in aliquots containing enough solution to do no more than three experiments (to limit the freeze/thaw cycle).

### Drug Plate Preparation:

### Dilution Drug Plate:

From the stock concentration, 8 serial dilutions (1:5) were made with DMSO.

Therefore, a total of 9 doses (stock plus 8 serial dilutions) were available for the dose response curve study.

### Working Drug Plate:

From the dilution drug plate, another dilution, across all doses, was made with either PBS or the cell culture media to be used in the screening with the cell lines.

Generally, about 5 μl was added to the 100 μl of cell culture during the treatment, and each dose was replicated in three wells.

Note: The final DMSO concentration in the treated well is 0.3% or less.

There is also three wells treated with 0.3% DMSO as vehicle control and (optional) three wells of cell culture treated with either PBS or media (no DMSO or compound additive).

### Screening Protocol:

Day −1: Plate cells in 100 μl volume in 96 well plate.
Note: Cell number seeded adjusted for growth characteristics, so that at time of assay for proliferation following three-day drug treatment, the control wells should still be in log-growth phase (sub-confluent).
   Day 0: Drug added to plate for treatment.
A time 0 plate was processed for the proliferation assay to establish a baseline reading at time when drug was added.
   Day 3: Process for proliferation assay (e.g. Cell-Titre Glo assay by Promega) with slight modification:
Prepare 1×CTG reagent (by diluting the 2×, as suggested by manufactuer, with PBS)
Remove media from plate (96-well), add 50 μl of 1×CTG per well.
Rotate the plate for 10 min at room temperature and read with BIO-TEK FLx800

### Data Calculation:

LBNL are following the protocols set up by the NCI/NIH DTP Human Tumor Cell Line Screen Process (http://dtp.nci.nih.gov/branches/btb/ivclsp.html) and summarized below.

Percentage growth inhibition is calculated as:

[(Ti−Tz)/(C−Tz)]×100 for concentrations for which Ti>/=Tz

[(Ti−Tz)/Tz]×100 for concentrations for which Ti<Tz.
C: control growth (vehicle control growth)
Tz: reading at time 0
Ti: test growth in the presence of drug at the nine concentration levels
   gIC₅₀: Growth inhibition of 50% is calculated from

[(Ti−Tz)/(C−Tz)]×100=50,
which is the drug concentration resulting in a 50% reduction in the net growth in control cells during the drug incubation.
   TGI: Total growth inhibition is calculated from

Ti=Tz.
The drug concentration resulting in total growth inhibition (down to the ‘time 0’ baseline).
   LC₅₀: Net loss of cell growth at 50% is calculated from

[(Ti−Tz)/Tz]×100=−50.
The concentration of drug resulting in a 50% reduction in the cell number at the end of the drug treatment as compared to that at the beginning (time 0) indicating a net loss of cells following treatment.

Values are calculated for each of these three parameters if the level of activity is reached; however, if the effect is not reached or is exceeded, the value for that parameter is expressed as greater or less than the maximum or minimum concentration tested.

A subset of the cell line set were characterised for their molecular subtype. This procedure for classifying these cell lines is described in Neve, R. M. et al. 2006. Cancer Cell 10: 515-27. Classifications were made based upon gene expression data.

Compound B was tested in a panel of 51 human breast cell lines. Cytotoxicity curves were generated and gIC₅₀s determined for all cells using two cell densities (Table 1). gIC₅₀s for Compound B across the 15 cell panel ranged from 1.1 to 398.1 nM. The overall median gIC₅₀ was 10.4 nM, while the average value was 28.9 nM. Only 16/51 (31%) tumor cell lines demonstrated a gIC₅₀>30 nM, while 7/51 (14%) had gIC₅₀s<3 nM.

The degree of responsiveness for each individual cell line was measured based upon gIC₅₀ calculations where lower values are more responsive the cell was to treatment with Compound B.

Mutation data for PIK3CA was available for 33 cell lines screened for responsiveness to Compound B. A total of 27% ( 9/33) cell lines had mutations of PIK3CA. A total of 49 cell lines were screened for HER2 status. Of these, 13/49 (27%) were considered HER2 amplified. These data are presented in Table 3.

TABLE 3
Activity of COMPOUND B in Human Cell Panel

| | Site/ | | | | HER2 | PIK3CA |
|---|---|---|---|---|---|---|
| Cell Line | Diagnosis | gIC₅₀ (nM) | TGI (nM) | LC₅₀ (nM) | Status | Mutation Status |
| 184A1 | Breast | 1.6 | 16.3 | 26742.4 | HER2− | NA |
| | epithelium | | | | | |
| 184B5 | Breast | 5.6 | 46.3 | >33000.0 | HER2− | NA |
| | epithelium | | | | | |
| 600MPE | Breast Tumor | 6.8 | 33.5 | 875.2 | HER2− | NA |
| AU565 | Breast Tumor | 7.2 | 6700.0 | >33000.0 | HER2+ | Wild Type |
| BT20 | Breast Tumor | 14.8 | 95.5 | 467.7 | HER2− | NA |
| BT474 | Breast Tumor | 4.8 | 20.1 | 119.1 | HER2+ | Mutant |
| BT483 | Breast Tumor | 1.5 | 8.5 | 1174.9 | HER2− | Mutant |
| BT549 | Breast Tumor | 42.7 | >33000.0 | >33000.0 | HER2− | Wild Type |
| CAMA1 | Breast Tumor | 89.1 | 239.9 | 537.0 | HER2− | Wild Type |
| HCC1143 | Breast Tumor | 34.0 | 344.0 | 19516.4 | HER2− | Wild Type |
| HCC1187 | Breast Tumor | 4.9 | 35.5 | 117.5 | HER2+ | NA |
| HCC1395 | Breast Tumor | 50.3 | 358.6 | 26742.4 | HER2− | Wild Type |
| HCC1419 | Breast Tumor | 1.5 | 5.2 | 514.4 | HER2− | Wild Type |
| HCC1428 | Breast Tumor | 16.2 | 158.5 | 5248.1 | HER2− | Wild Type |
| HCC1500 | Breast Tumor | 75.9 | 371.5 | 6500.0 | HER2− | Wild Type |
| HCC1569 | Breast Tumor | 16.3 | 67.4 | >33000.0 | HER2+ | Wild Type |
| HCC1806 | Breast Tumor | 26.2 | 375.1 | 2819.7 | NA | Wild Type |
| HCC1937 | Breast Tumor | 25.4 | 321.7 | 33000.0 | HER2− | Wild Type |
| HCC1954 | Breast Tumor | 14.3 | 32.3 | 54.2 | HER2− | Mutant |
| HCC202 | Breast Tumor | 1.1 | 2.6 | 21.4 | HER2+ | Mutant |
| HCC2185 | Breast Tumor | 3.2 | 10.0 | 131.8 | NA | NA |
| HCC3153 | Breast Tumor | 38.9 | 281.8 | 35481.3 | NA | NA |
| HCC38 | Breast Tumor | 55.0 | 158.5 | 354.8 | HER2− | Wild Type |
| HCC70 | Breast Tumor | 4.9 | 26.8 | 118.2 | HER2− | Wild Type |
| Hs578T | Breast Tumor | 67.4 | 4556.2 | >33000.0 | HER2− | Wild Type |
| LY2 | Breast Tumor | 11.0 | 30.2 | 67.9 | HER2− | NA |
| M4A4 | Breast Tumor | 30.2 | 281.8 | >33000.0 | HER2− | NA |
| MCF10A | Breast | 7.2 | 467.7 | 6700.0 | HER2− | NA |
| | epithelium | | | | | |
| MCF10F | Breast | 2.4 | 14.3 | 98.7 | HER2− | NA |
| | epithelium | | | | | |
| MCF12A | Breast | 7.4 | 178.5 | 2756.8 | HER2− | Wild Type |
| | epithelium | | | | | |
| MCF7 | Breast Tumor | 7.3 | 22.0 | 149.1 | HER2− | Mutant |
| MDAMB134 | Breast Tumor | 13.2 | 60.3 | 223.9 | HER2− | Wild Type |
| MDAMB157 | Breast Tumor | 398.1 | 3981.1 | >33000.0 | HER2− | Wild Type |
| MDAMB175VII | Breast Tumor | 4.4 | 13.5 | 501.2 | HER2+ | Wild Type |
| MDAMB231 | Breast Tumor | 109.6 | >33000.0 | >33000.0 | HER2− | Wild Type |
| MDAMB361 | Breast Tumor | 1.7 | 4.9 | 26.9 | HER2+ | Mutant |
| MDAMB415 | Breast Tumor | 20.8 | 117.7 | 527.5 | HER2− | Wild Type |
| MDAMB436L | Breast Tumor | 13.2 | >33000.0 | >33000.0 | HER2− | NA |
| MDAMB453 | Breast Tumor | 7.8 | 54.9 | 161.8 | HER2+ | Mutant |
| MDAMB468D | Breast Tumor | 31.6 | 537.0 | 2238.7 | NA | NA |
| MX-1 | Breast Tumor | 5.2 | 20.4 | 251.2 | HER2− | NA |
| SKBR3 | Breast Tumor | 3.2 | 199.5 | >33000.0 | HER2+ | Wild Type |
| SUM1315MO2 | Breast Tumor | 10.0 | 158.3 | 8881.8 | HER2− | Wild Type |
| SUM149PT | Breast Tumor | 20.6 | 862.2 | 2596.6 | HER2− | NA |
| SUM159PT | Breast Tumor | 10.4 | 940.6 | >33000.0 | HER2− | Mutant |
| SUM225CWN | Breast Tumor | 1.6 | 14.8 | 436.5 | HER2− | NA |
| SUM229PE | Breast Tumor | 2.5 | 17.4 | 125.9 | HER2− | NA |
| T47D | Breast Tumor | 3.7 | 19.1 | 1000.0 | HER2− | Mutant |
| T47DKBluc | Breast Tumor | 15.4 | 74.3 | 9501.7 | HER2− | NA |
| UACC812 | Breast Tumor | 4.9 | 24.0 | 955.0 | HER2+ | Wild Type |
| UACC893D | Breast Tumor | 8.8 | 20.1 | 116.7 | HER2+ | NA |
| ZR751 | Breast Tumor | 10.5 | 30.2 | 248.3 | HER2+ | Wild Type |
| ZR75B | Breast Tumor | 4.5 | 43.4 | >33000.0 | HER2+ | NA |

Table 1 Key:
Cell Line = Tumor-derived cell line
Site/Diagnosis = Site and Diagnosis of tissue
gIC₅₀ = Concentration of compound required to cause 50% growth inhibition
TGI = Total Growth Inibition
LC50 = drug concentration resulting in a 50% reduction in the net growth in control cells during the drug incubation.
HER2 Status = DNA copy number status of the HER2 Gene.
HER2+ = Amplified,
HER2− = Not Amplified,
NA = Data not available
PIK3CA Mutation Status = Mutant = PIK3CA mutant cell line;
Wild Type = Cell line with no PIK3CA mutation;
‘NA’ = Data not available for cell line

While the preferred embodiments of the invention are illustrated by the above, it is to be understood that the invention is not limited to the precise instructions herein disclosed and that the right to all modifications coming within the scope of the following claims is reserved.

We claim:

1. A method of treating a human with cancer comprising detecting at least one mutation in a PIK3CA gene or at least one mutant protein encoded by said PIK3CA gene from at least one first sample from said human and administering to said human an effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition if said at least one sample has at least one mutant PI3K protein or a mutation in the PIK3CA gene.

2. The method of claim 1, wherein said mutation in the PIK3CA gene is a somatic mutation.

3. The method of claim 1, wherein said mutation in the PIK3CA gene is selected from: 3140A>G, 1633G>A, 1624G>A, 3140A>T, 1634A>C, 1634A>G, 1636C>A, and 333G>C.

4. The method of claim 1, wherein said at least one mutation in the protein encoded by the PIK3CA gene is selected from: H1047L, H1047R, Q546K, E545A, M1043I, E545D, E545K, P539R, K111N, P449T, and E542K.

5. (canceled)

6. The method of claim 1, wherein said cancer is selected from: breast, colon, renal cell carcinoma, lung, liver, bladder, melanoma, and lymphatic.

7. (canceled)

8. The method of claim 1, further comprising determining whether said human has a tumor with three or more copies of the HER2 gene.

9. (canceled)

10. The method of claim 1, further comprising determining whether said human has a tumor that overexpresses Her2 protein, a fragment thereof, or both.

11. The method of claim 1, further comprising determining whether said sample has a mutation in a KRAS gene, and administering to said human an effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition if said sample does not have a mutation in a KRAS gene.

12. The method of claim 1, further comprising determining the RAS protein mutation status from at least one second sample from said human.

13. The method claim 12, wherein said first sample and said second sample are independently selected from the group consisting of a tumor sample and a blood sample.

14-16. (canceled)

17. The method of claim 12, wherein said Ras protein is KRAS.

18. The method of claim 12, wherein said mutation in said Ras protein is selected from: G12S, G12V, G12D, G12A, G12C, G12R, G13A, G13D, Q61K, Q61R, E76G, E76K, E76Q, and A146T.

19. The method of claim 17, wherein said Ras protein is KRAS and the mutation in KRAS is selected from: G12S, G12V, G12D, G12A, G12C, G12R, and G13A.

20. A method of treating a patient with cancer comprising detecting the number of copies of the Her2 gene in at least one tumor cell or the amount of Her2/neu receptor expressed by said tumor cell from said patient and administering a therapeutically effective amount of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition if said tumor cell has 3 or more copies of the Her2 gene or if said tumor cell expresses a greater amount of a Her 2 gene product than a non-tumor cell.

21. The method of claim 18, wherein said tumor cell is selected from: breast, bladder, pancreatic, lung, colon, melanoma and lymphoid.

22. The method of claim 1, wherein said method comprises detecting at least one mutation in a PIK3CA gene, and wherein said detecting comprises genotyping at least one tumor cell from said human for at least one mutation in a PIK3CA gene, and if at least one mutation in a PIK3CA gene is detected administering at least one dose of 2,4-difluoro-N-{2-(methyloxy)-5-[4-(4-pyridazinyl)-6-quinolinyl]-3-pyridinyl}benzenesulfonamide or a pharmaceutically acceptable salt thereof in a pharmaceutical composition.

23-27. (canceled)

28. The method of claim 22, further comprising administering at least one dose of a second anti-neoplastic agent.

29-30. (canceled)
